(19) United States
(12) Patent Application Publication
(10) Pub. No.: US 2023/0157624 A1
(43) Pub. Date: May 25, 2023

(54) INFORMATION PROCESSOR AND INFORMATION PROCESSING PROGRAM

(71) Applicant: SONY GROUP CORPORATION, TOKYO (JP)

(72) Inventor: YUTA KONDO, TOKYO (JP)

(21) Appl. No.: 17/995,652

(22) PCT Filed: Apr. 7, 2021
(86) PCT No.: PCT/JP2021/014823

§ 371 Date: Oct. 6, 2022

(30) Foreign Application Priority Data
Apr. 14, 2020 (JP) 2020-072405

(51) Int. Cl. A61B 5/00; A61B 5/0531; A61B 5/0537

(52) U.S. Cl. CPC A61B 5/4266; A61B 5/0531; A61B 5/7246; A61B 5/0537

(57) ABSTRACT

An information processor according to an aspect of the present disclosure includes an acquisition section and a generation section. The acquisition section acquires external sweat amount data, on an amount of sweat oozing out of an epidermis, generated on a basis of an output of a first sensor section, and internal sweat amount data, on an amount of sweat inside the epidermis and a dermis, generated on a basis of an output of a second sensor section. The generation section generates sweat amount data on a basis of the external sweat amount data and the internal sweat amount data.

### TECHNICAL FIELD

The present disclosure relates to an information processor and an information processing program.

### BACKGROUND ART

In recent years, various measurement techniques for determining biological information have been studied. For example, an electrodermal response (GSR: Galvanic Skin Response) has been heretofore utilized as one of the biological information. An electric activity of the skin of a user, which is also available as the biological information as in the GSR, is also collectively referred to as an electrodermal activity (EDA: Electro-Dermal Activity). In addition, a skin potential activity (SPA: Skin Potential Activity) is also included in the EDA. The EDA has been widely used, for example, as a method for detecting an activity of an autonomic nervous system of a user.

As a measure to reduce a body motion noise superimposed on a measurement signal obtained by an EDA sensor, for example, an inventions described in PTL 1 has been known.

### CITATION LIST

### Patent Literature

PTL 1: Japanese Unexamined Patent Application Publication No. 2020-10803

### SUMMARY OF THE INVENTION

Incidentally, PTL 1 describes utilization of a pressure sensor to reduce a body motion noise. However, due to a structural constraint on the pressure sensor, a reduction effect of the body motion noise is limitative. It is therefore desirable to provide an information processor and an information processing program that make it possible to reduce a body motion noise more effectively.

An information processor according to a first aspect of the present disclosure includes an acquisition section and a generation section. The acquisition section acquires external sweat amount data, on an amount of sweat oozing out of an epidermis, generated on a basis of an output of a first sensor section, and internal sweat amount data, on an amount of sweat inside the epidermis and a dermis, generated on a basis of an output of a second sensor section. The generation section generates sweat amount data on a basis of the external sweat amount data and the internal sweat amount data.

In the information processor according to the first aspect of the present disclosure, sweat amount data is generated on the basis of external sweat amount data and internal sweat amount data generated on the basis of outputs of the first sensor section and the second sensor section. This makes it possible, for example, to use the internal sweat amount data as reference data of the external sweat amount data and to use the external sweat amount data as reference data of the internal sweat amount data. As a result, it is possible to detect and reduce a body motion noise from the external sweat amount data or the internal sweat amount data without using an output of the pressure sensor as the reference data.

An information processing program according to a second aspect of the present disclosure causes a computer to:

(1) acquire external sweat amount data on an amount of sweat oozing out of an epidermis and internal sweat amount data on an amount of sweat inside the epidermis and a dermis, the external sweat amount data being generated on a basis of an output of a first sensor section, the internal sweat amount data being generated on a basis of an output of a second sensor; and
(2) generate sweat amount data on a basis of the external sweat amount data and the internal sweat amount data.

In the information processing program according to the second aspect of the present disclosure, sweat amount data is generated on the basis of external sweat amount data and internal sweat amount data generated on the basis of outputs of the first sensor section and the second sensor section. This makes it possible, for example, to use the internal sweat amount data as reference data of the external sweat amount data and to use the external sweat amount data as reference data of the internal sweat amount data. As a result, it is possible to detect and reduce a body motion noise from the external sweat amount data or the internal sweat amount data without using an output of the pressure sensor as the reference data.

### BRIEF DESCRIPTION OF THE DRAWINGS

FIG. 1 is a diagram illustrating an example of a schematic configuration of a biological information processor according to an embodiment of the present disclosure.

FIG. 2 is a diagram illustrating an example of a cross-sectional configuration of a vicinity of a biological surface with which a sensor unit in FIG. 1 is brought into contact.

FIG. 3 is a diagram illustrating a configuration example of a portion of the sensor unit in FIG. 1.

FIG. 4 is a diagram illustrating a configuration example of a portion of the sensor unit in FIG. 1.

FIG. 5 is a diagram illustrating a configuration example of a portion of the sensor unit in FIG. 1.

FIG. 6 is a diagram illustrating an example of functional blocks of a basic measurement section in FIG. 1.

FIG. 7 is a diagram illustrating an example of a determination table to be used in a reliability determination section in FIG. 1.

FIG. 8 is a diagram illustrating an example of a processing procedure in the reliability determination section in FIG. 1.

FIG. 9 illustrates a modification example of the processing procedure in the reliability determination section in FIG. 1.

FIG. 10 is an explanatory diagram of temporal changes in an internal sweat amount and an external sweat amount.

FIG. 11 illustrates a modification example of a processing procedure in a sweat intensity estimation section in FIG. 1.

FIG. 12 illustrates a modification example of a schematic configuration of the biological information processor in FIG. 1.

FIG. 13 is a diagram illustrating a state in which the biological information processor in each of FIGS. 1 and 12 is built in a wearable apparatus.

FIG. 14 is a diagram illustrating a state in which the biological information processor in each of FIGS. 1 and 12 is built in the wearable apparatus.

FIG. 15 is a diagram illustrating a state in which the wearable apparatus in FIGS. 13 and 14 is coupled to a terminal apparatus via a network.

FIG. 16 is a diagram illustrating an example in which some of functions of the biological information processor in FIG. 1 are provided in a server apparatus.

FIG. 17 is a diagram illustrating an example in which some of functions of the biological information processor in FIG. 12 are provided in the server apparatus.

FIG. 18 is a diagram illustrating an example in which some of functions of the biological information processor in FIG. 1 are provided in the server apparatus.

FIG. 19 is a diagram illustrating an example in which some of functions of the information processor in FIG. 12 are provided in the server apparatus.

FIG. 20 is a diagram illustrating an example in which some of functions of the information processor in FIG. 1 are provided in the server apparatus.

### MODES FOR CARRYING OUT THE INVENTION

Hereinafter, description is given in detail of embodiments for carrying out the present disclosure with reference to the drawings. It is to be noted that the description is given in the following order.

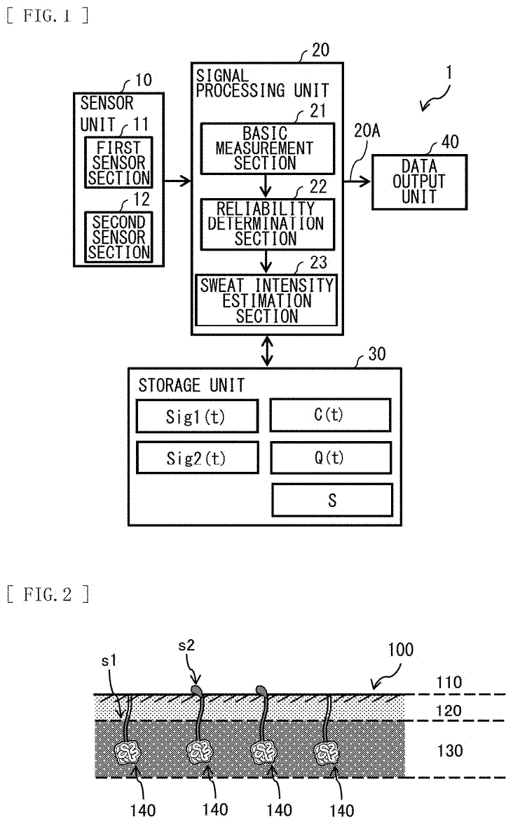

### 1. Embodiment (FIGS. 1 to 11)

An example in which a sensor that detects external sweating and a sensor that detects internal sweating are provided

### 2. Modification Example

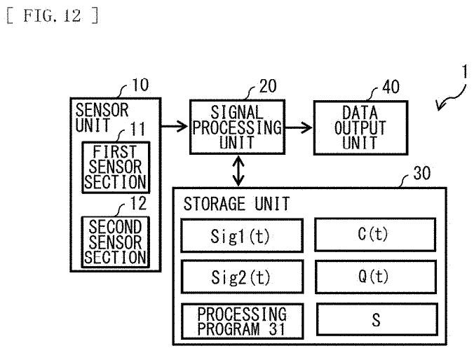

An example in which functions of a signal processing unit are implemented by a program (FIG. 12)

### 3. Application Example

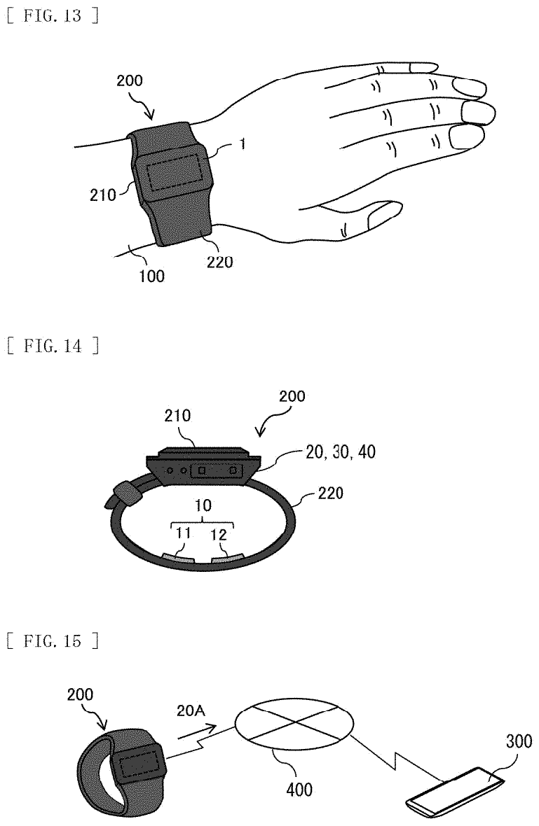

An example in which a biological information processor is provided in a wearable apparatus (FIGS. 13 to 20)

### 1. Embodiment

Human beings have a function to sweat on a body surface as a response of an autonomic nervous system with respect to an environmental change. Examples of the sweating include thermal sweating to adjust a body temperature in a hot environment or during exercise, mental sweating at the time when subjected to a mental stimulus such as a mental tension or an emotional change, gustatory sweating at the time when eating something hot and spicy, and the like.

There is biological measurement called sweating measurement to acquire a change due to this sweating on the body surface. The sweating measurement is performed by using a sweat sensor. It is a common technique, in the sweating measurement, to place at least two or more electrodes on a body surface and apply a voltage or flow a current between the electrodes to acquire a change in impedance (or a change in conductance) between the electrodes due to an action of sweating on the body surface.

In the sweating measurement, active sweat glands and a sweat amount on a current path affect measurement results. In daily use, a wrist on which a wristwatch or the like is worn is considered to be useful as a measurement site. However, the wrist has less active sweat glands and less changes in the sweat amount than those of a finger or a palm. In addition, there are individual differences in the distribution of active sweat glands, thus making it difficult to specify locations thereof. This makes it difficult to measure a skin conductance response, which is a detection target of the mental sweating.

Examples of a method to solve the above issue include a method of increasing the area of electrodes and the detection rate of active sweat glands to thereby increase a signal intensity for detection. However, such a method also increases a region subjected to a change in contact between the electrode and the skin, thus making a body motion noise due to the change in contact between the electrode and the skin more likely to occur depending on a state of the worn sweat sensor. Examples of a measure to reduce the body motion noise include an invention described in PTL 1 mentioned above. However, the method described in PTL 1 utilizes a pressure sensor to reduce the body motion noise, and thus a reduction effect of the body motion noise is limitative due to a structural constraint on the pressure sensor. The present disclosure proposes a method of reducing the body motion noise more effectively without using the pressure sensor.

### [Configuration]

FIG. 1 illustrates an example of a schematic configuration of a biological information processor 1 according to an embodiment of the present disclosure. The biological information processor 1 is a device that measures mental sweating by means of an electric or optical method, or the like. The biological information processor 1 includes a sensor unit 10, a signal processing unit 20, a storage unit 30, and a data output unit 40. FIG. 2 illustrates an example of a cross-sectional configuration of a vicinity of a surface of a biological body 100 (a biological surface 110) with which the sensor unit 10 is brought into contact.

In the biological body 100, the biological surface 110 is covered with an epidermis 120, and a dermis 130 is formed below the epidermis 120 with a basement membrane interposed therebetween. A large number of sweat glands 140 are formed in the epidermis 120 and the dermis 130. External sweat s2 oozes to the biological surface 110 from the sweat gland 140, and internal sweat s1 that does not ooze to the biological surface 110 is present inside the epidermis 120 and the dermis 130. The external sweat s2 is sweat that oozes out of the epidermis 120. The internal sweat s1 is sweat inside the epidermis 120 and the dermis 130.

The sensor unit 10 includes two types of sensor sections (a first sensor section 11 and a second sensor section 12). The first sensor section 11 is an electric sweat sensor that electrically detects an amount of sweat (external sweat amount) oozing out of the epidermis 120 and outputs first sensor data Sig1(t) obtained thereby. The second sensor section 12 is an optical sweat sensor that optically detects an amount of sweat (internal sweat amount) inside the epidermis 120 and the dermis 130 and outputs second sensor data Sig2(t) obtained thereby. The first sensor section 11 and the second sensor section 12 are in contact with the same location or a location equivalent thereto of the biological surface 110. The “equivalent location” refers to a location that allows for substantially equal measurement as compared with a case where the first sensor section 11 and the second sensor section 12 are placed in the same location of the biological surface 110.

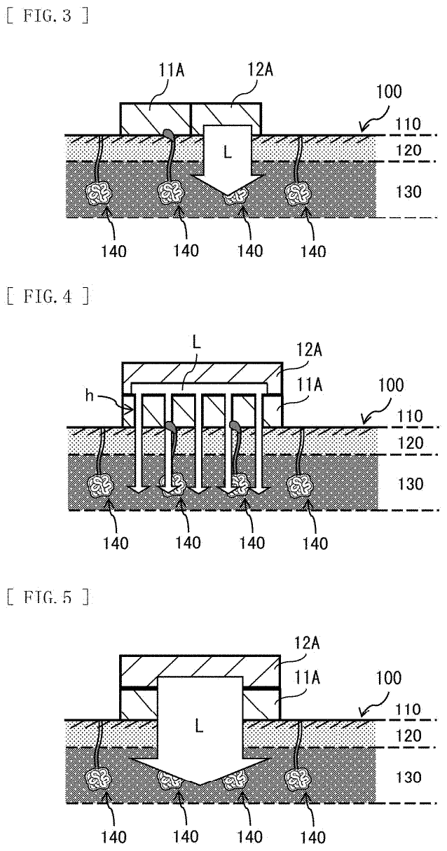

For example, as illustrated in FIG. 3, the first sensor section 11 includes a detection electrode 11A, and, upon measurement, brings the detection electrode 11A into contact with the biological surface 110 to flow a predetermined current to the epidermis 120, thereby detecting the external sweat amount. The detection electrode 11A is configured by an electrically-conductive metal layer, for example. For example, as illustrated in FIG. 3, the second sensor section 12 includes a light source 12A. The light source 12A irradiates the “equivalent location” of the biological surface 110 with light L in a wavelength band reaching the dermis 130, and the second sensor section 12 detects reflected light thereof to thereby detect the internal sweat amount.

For example, the detection electrode 11A may be provided with a plurality of openings h as illustrated in FIG. 4. The plurality of openings h is provided to transmit the light L. At this time, for example, as illustrated in FIG. 4, the light source 12A is disposed on the detection electrode 11A. The light source 12A disposed on the detection electrode 11A irradiates the “same location” of the biological surface 110 with the light L in the wavelength band reaching the dermis 130 via the plurality of openings h.

The detection electrode 11A may be configured by a light-transmissive electrically-conductive material (e.g., ITO, etc.) that transmits the light L. In this case, the light L is transmitted through the detection electrode 11A even when the detection electrode 11A is not provided with the plurality of openings h. Accordingly, for example, as illustrated in FIG. 5, the light source 12A is disposed on the detection electrode 11A, and the light source 12A disposed on the detection electrode 11A irradiates the “same location” of the biological surface 110 with the light L in the wavelength band reaching the dermis 130 via the detection electrode 11A.

The storage unit 30 is, for example, a volatile memory such as a DRAM (Dynamic Random Access Memory), or a non-volatile memory such as an EEPROM (Electrically Erasable Programmable Read-Only Memory) or a flush memory. The storage unit 30 stores sensor data (first sensor data Sig1(t) and second sensor data Sig2(t)) obtained from the sensor unit 10, and data (external sweat amount data C(t), internal sweat amount data Q(t), and sweat intensity S) generated by the signal processing unit 20.

The signal processing unit 20 includes, for example, a basic measurement section 21, a reliability determination section 22, and a sweat intensity estimation section 23, as illustrated in FIG. 1.

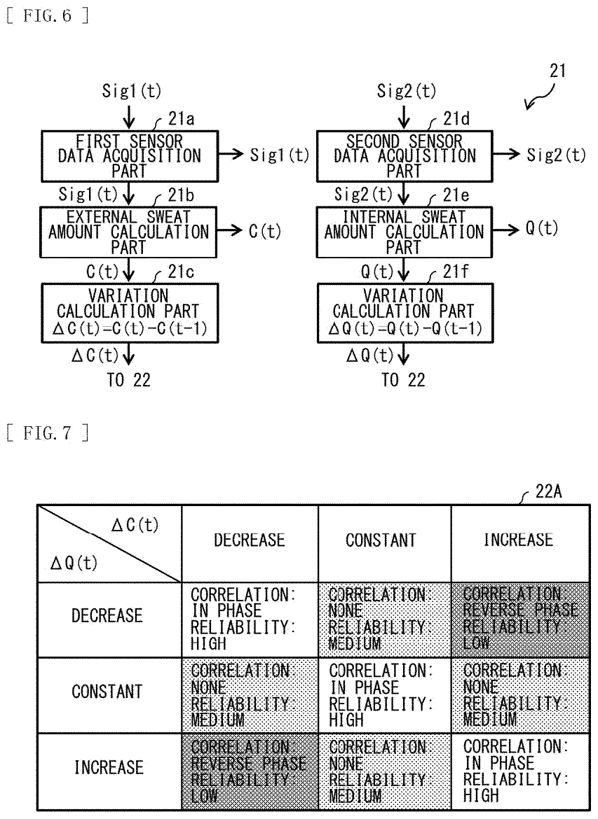

As illustrated in FIG. 6, the basic measurement section 21 includes, for example, a first sensor data acquisition part 21a that acquires the analog first sensor data Sig1(t), and an external sweat amount calculation part 21b that generates the external sweat amount data C(t) on the basis of the first sensor data Sig1(t). The external sweat amount calculation part 21b includes, for example, an IV converter that performs IV-conversion of the first sensor data Sig1(t) which is a current signal, an amplifier that amplifies a voltage signal obtained by the IV-conversion, and a filter that reduces an unnecessary frequency component included in the amplified voltage signal. The external sweat amount calculation part 21b outputs, to a variation calculation part 21c, the external sweat amount data C(t) which is a voltage signal obtained by pieces of processing performed by the IV converter, the amplifier, and the filter, for example.

As illustrated in FIG. 6, the basic measurement section 21 further includes, for example, the variation calculation part 21c. The variation calculation part 21c calculates first difference data ΔC(t) (=C(t)−C(t−1)), which is a difference between two pieces of data having detection times different from each other, among the external sweat amount data C(t). The variation calculation part 21c outputs the calculated first difference data ΔC(t) to the reliability determination section 22.

As illustrated in FIG. 6, the basic measurement section 21 further includes, for example, a second sensor data acquisition part 21d that acquires the analog second sensor data Sig2(t), and an internal sweat amount calculation part 21e that generates the internal sweat amount data Q(t) on the basis of the second sensor data Sig2(t). The internal sweat amount calculation part 21e includes, for example, an IV converter that performs IV-conversion of the second sensor data Sig2(t) which is a current signal, an amplifier that amplifies a voltage signal obtained by the IV-conversion, and a filter that reduces an unnecessary frequency component included in the amplified voltage signal. The internal sweat amount calculation part 21e outputs, to a variation calculation part 21f, the internal sweat amount data Q(t) which is a voltage signal obtained by pieces of processing performed by the IV converter, the amplifier, and the filter, for example.

As illustrated in FIG. 6, the basic measurement section 21 further includes, for example, the variation calculation part 21f. The variation calculation part 21f calculates second difference data ΔQ(t) (=Q(t)−Q(t−1)), which is a difference between two pieces of data having detection times different from each other, among the internal sweat amount data Q(t). The variation calculation part 21f outputs the calculated second difference data ΔQ(t) to the reliability determination section 22.

FIG. 7 illustrates an example of a determination table 22A to be used in the reliability determination section 22. The determination table 22A specifies a correlation between the first difference data ΔC(t) and the second difference data ΔQ(t) and reliability thereof. For example, in a case where both the first difference data ΔC(t) and the second difference data ΔQ(t) decrease (i.e., being negative values), the first difference data ΔC(t) and the second difference data ΔQ(t) have a correlation of an in phase with respect to each other. At this time, it can be said that the reliability of each of the first difference data ΔC(t) and the second difference data ΔQ(t) is high. In addition, for example, in a case where both the first difference data ΔC(t) and the second difference data ΔQ(t) are constant (i.e., being values of roughly zero), the first difference data ΔC(t) and the second difference data ΔQ(t) have a correlation of an in phase with respect to each other. At this time, it can be said that the reliability of each of the first difference data ΔC(t) and the second difference data ΔQ(t) is high. In addition, for example, in a case where both the first difference data ΔC(t) and the second difference data ΔQ(t) increase (i.e., being positive values), the first difference data ΔC(t) and the second difference data ΔQ(t) have a correlation of an in phase with respect to each other. At this time, it can be said that the reliability of each of the first difference data ΔC(t) and the second difference data ΔQ(t) is high.

In addition, for example, in a case where the first difference data ΔC(t) is constant (i.e., being a value of roughly zero) and the second difference data ΔQ(t) decreases (i.e., being a negative value), there is no correlation between the first difference data ΔC(t) and the second difference data ΔQ(t). At this time, it can be said that the reliability of each of the first difference data ΔC(t) and the second difference data ΔQ(t) is medium. In addition, for example, in a case where the first difference data ΔC(t) decreases (i.e., being a negative value) and the second difference data ΔQ(t) is constant (i.e., being a value of roughly zero), there is no correlation between the first difference data ΔC(t) and the second difference data ΔQ(t). At this time, it can be said that the reliability of each of the first difference data ΔC(t) and the second difference data ΔQ(t) is medium.

In addition, for example, in a case where the first difference data ΔC(t) increases (i.e., being a positive value) and the second difference data ΔQ(t) is constant (i.e., being a value of roughly zero), there is no correlation between the first difference data ΔC(t) and the second difference data ΔQ(t). At this time, it can be said that the reliability of each of the first difference data ΔC(t) and the second difference data ΔQ(t) is medium. In addition, for example, in a case where the first difference data ΔC(t) is constant (i.e., being a value of roughly zero) and the second difference data ΔQ(t) increases (i.e., being a positive value), there is no correlation between the first difference data ΔC(t) and the second difference data ΔQ(t). At this time, it can be said that the reliability of each of the first difference data ΔC(t) and the second difference data ΔQ(t) is medium.

In addition, for example, in a case where the first difference data ΔC(t) increases (i.e., being a positive value) and the second difference data ΔQ(t) decreases (i.e., being a negative value), the first difference data ΔC(t) and the second difference data ΔQ(t) have a relationship of a reverse phase with respect to each other. At this time, it can be said that the reliability of each of the first difference data ΔC(t) and the second difference data ΔQ(t) is low. In addition, for example, in a case where the first difference data ΔC(t) decreases (i.e., being a negative value) and the second difference data ΔQ(t) increases (i.e., being a positive value), the first difference data ΔC(t) and the second difference data ΔQ(t) have a relationship of a reverse phase with respect to each other. At this time, it can be said that the reliability of each of the first difference data ΔC(t) and the second difference data ΔQ(t) is low.

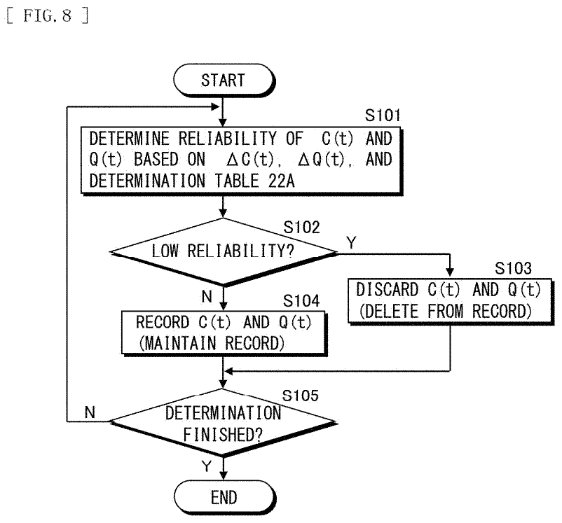

For example, the reliability determination section 22 uses the determination table 22A to evaluate the reliability of each of the external sweat amount data C(t) and the internal sweat amount data Q(t), and, on the basis of a result of the evaluation, records/discards data corresponding to the reliability in a procedure illustrated in FIG. 8, for example.

The reliability determination section 22 determines the reliability of the external sweat amount data C(t) on the basis of the second difference data ΔQ(t), for example (step S101). The reliability determination section 22 determines the reliability of the internal sweat amount data Q(t) on the basis of the first difference data ΔC(t), for example (step S101).

For example, in a case where the first difference data ΔC(t) and the second difference data ΔQ(t) include a correlation of a reverse phase with respect to each other, the reliability determination section 22 gives a low evaluation to each reliability of portions in the correlation of the reverse phase with respect to each other, among the external sweat amount data C(t) and the internal sweat amount data Q(t). For example, in a case where the first difference data ΔC(t) and the second difference data ΔQ(t) include a correlation of an in phase with respect to each other, the reliability determination section 22 gives a high evaluation to each reliability of portions in the correlation of the in phase with respect to each other, among the external sweat amount data C(t) and the internal sweat amount data Q(t). For example, a calculation such as normalized cross-correlation may be used to determine whether the correlation is an in phase or a reverse phase.

The reliability determination section 22 performs processing on the external sweat amount data C(t) on the basis of a result of the evaluation of the reliability of the external sweat amount data C(t), for example, and sets the resulting data as sweat amount data 20A (to be included in the sweat amount data 20A). The reliability determination section 22 performs processing on the internal sweat amount data Q(t) on the basis of a result of the evaluation of the reliability of the internal sweat amount data Q(t), for example, and sets the resulting data as the sweat amount data 20A (to be included in the sweat amount data 20A). That is, the reliability determination section 22 generates the sweat amount data 20A on the basis of the external sweat amount data C(t) and the internal sweat amount data Q(t).

For example, in a case where the reliability of the first difference data ΔC(t) is low (step S102; Y), the reliability determination section 22 discards the external sweat amount data C(t) corresponding to the first difference data ΔC(t), or deletes the external sweat amount data C(t) corresponding to the first difference data ΔC(t) from the storage unit 30 (step S103). At this time, the reliability determination section 22 excludes the external sweat amount data C(t) corresponding to the first difference data ΔC(t) from the sweat amount data 20A.

In addition, for example, in a case where the reliability of the second difference data ΔQ(t) is low (step S102; Y), the reliability determination section 22 discards the internal sweat amount data Q(t) corresponding to the second difference data ΔQ(t), or deletes the internal sweat amount data Q(t) corresponding to the second difference data ΔQ(t) from the storage unit 30 (step S103). At this time, the reliability determination section 22 excludes the internal sweat amount data Q(t) corresponding to the second difference data ΔQ(t) from the sweat amount data 20A.

For example, in a case where the reliability of the first difference data ΔC(t) is not low (step S102; N), i.e., in a case where the reliability of the first difference data ΔC(t) is high or medium, the reliability determination section 22 records the external sweat amount data C(t) corresponding to the first difference data ΔC(t) in the storage unit 30, or maintains the recording of the external sweat amount data C(t) corresponding to the first difference data ΔC(t) recorded in the storage unit 30 (step S104). At this time, the reliability determination section 22 sets the external sweat amount data C(t) corresponding to the first difference data ΔC(t) as the sweat amount data 20A (to be included in the sweat amount data 20A).

For example, in a case where the reliability of the second difference data ΔQ(t) is not low (step S102; N), i.e., in a case where the reliability of the second difference data ΔQ(t) is high or medium, the reliability determination section 22 records the internal sweat amount data Q(t) corresponding to the second difference data ΔQ(t) in the storage unit 30, or maintains the recording of the internal sweat amount data Q(t) corresponding to the second difference data ΔQ(t) recorded in the storage unit 30 (step S104). At this time, the reliability determination section 22 sets the internal sweat amount data Q(t) corresponding to the second difference data ΔQ(t) as the sweat amount data 20A (to be included in the sweat amount data 20A).

In a case where the determination of the reliability is not finished (step S105; N), the reliability determination section 22 continues to execute step S101; in a case other than that (step S105; Y), the reliability determination section 22 finishes the determination of the reliability.

For example, the reliability determination section 22 may use the determination table 22A to evaluate the reliability of each of the external sweat amount data C(t) and the internal sweat amount data Q(t), and, on the basis of a result of the evaluation, may record data corresponding to the reliability in a procedure illustrated in FIG. 9, for example.

The reliability determination section 22 generates first reliability evaluation data Ec(t) on the external sweat amount data C(t) on the basis of a result of the evaluation of the reliability of the external sweat amount data C(t), for example (step S106). The first reliability evaluation data Ec(t) includes, for example, data (t) on the result of the evaluation of the reliability of the external sweat amount data C(t). At this time, the reliability determination section 22 sets each of the external sweat amount data C(t) and the first reliability evaluation data Ec(t) as the sweat amount data 20A (to be included in the sweat amount data 20A).

The reliability determination section 22 generates second reliability evaluation data Eq(t) corresponding to the internal sweat amount data Q(t) on the basis of a result of the evaluation of the reliability of the internal sweat amount data Q(t), for example (step S106). The first reliability evaluation data Ec(t) includes, for example, the data (t) on the result of the evaluation of the reliability of the internal sweat amount data Q(t). At this time, the reliability determination section 22 sets each of the internal sweat amount data Q(t) and the second reliability evaluation data Eq(t) as the sweat amount data 20A (to be included in the sweat amount data 20A).

Next, description is given of the sweat intensity estimation section 23. In a case where the reliability is high and both the first difference data ΔC(t) and the second difference data ΔQ(t) increase (i.e., being positive values) in the reliability determination section 22, the sweat intensity estimation section 23 performs sweat intensity estimation.

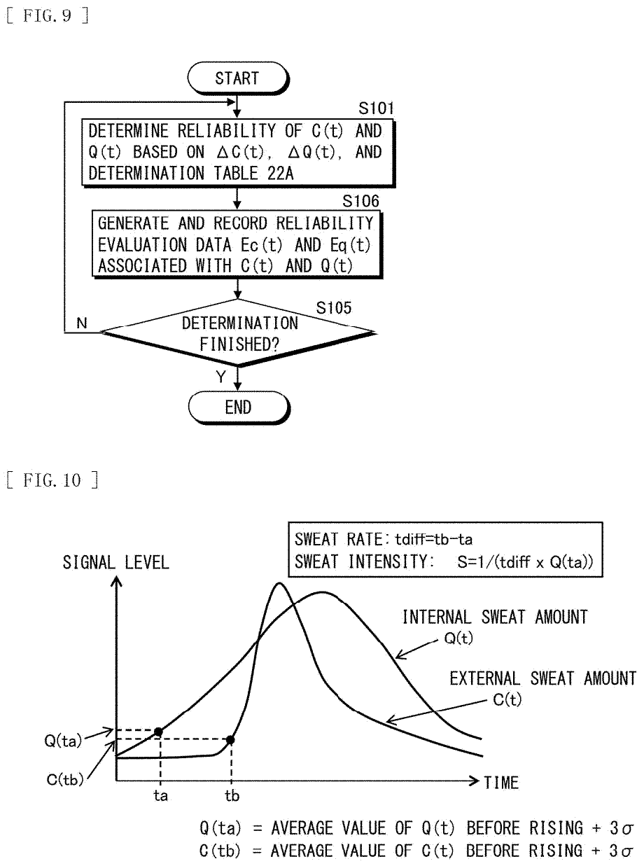

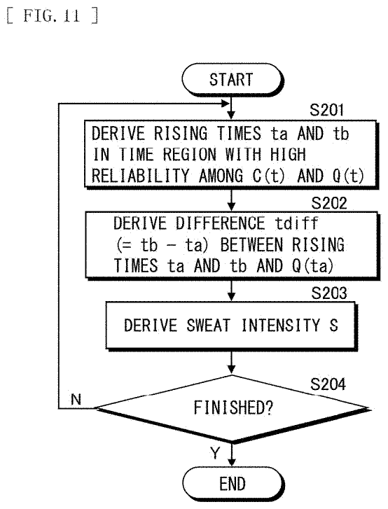

FIG. 10 illustrates an example of temporal changes in the internal sweat amount data Q(t) and the external sweat amount data C(t). FIG. 11 illustrates an example of a procedure to estimate sweat intensity in the sweat intensity estimation section 23.

First, the sweat intensity estimation section 23 derives rising times ta and tb in a time region with high reliability among the internal sweat amount data Q(t) and the external sweat amount data C(t) (step S201). Here, the rising time ta refers to rising time in the time region with high reliability of the internal sweat amount data Q(t). Internal sweat amount data Q(ta) is, for example, an average value of waveforms before rising in the time region with high reliability of the internal sweat amount data Q(t)+3σ. The sweat intensity estimation section 23 detects, as the rising time ta, time when the internal sweat amount data Q(t) gradually rises to exceed the internal sweat amount data Q(ta). The rising time tb refers to rising time in the time region with high reliability of the external sweat amount data C(t). External sweat amount data C(tb) is, for example, an average value of waveforms before rising in the time region with high reliability of the external sweat amount data C(t)+3σ. The sweat intensity estimation section 23 detects, as the rising time tb, time when the external sweat amount data C(t) gradually rises to exceed the external sweat amount data C(tb). The rising times ta and tb may be derived by a calculation method different from those described above.

Next, the sweat intensity estimation section 23 derives a difference tdiff(=tb−ta) between the rising times ta and tb and the internal sweat amount data Q(ta) (step S202). The difference tdiff(=tb−ta) is correlated with a rate (sweat rate) at which sweat is excreted from the inside of the sweat gland 140 to the outside. The internal sweat amount data Q(ta) is an amount of sweat accumulated (accumulated sweat amount) inside the sweat gland 140 at the rising time ta. From the graph in FIG. 10, as the accumulated sweat amount becomes larger, the difference tdiff becomes smaller, and, as the accumulated sweat amount becomes smaller, the difference tdiff becomes larger. For this reason, the sweat intensity S using the difference tdiff and the internal sweat amount data Q(ta) may be a new index in the sweating measurement. Therefore, the sweat intensity estimation section 23 derives the sweat intensity S (step S203). The sweat intensity S is represented by 1/(tdiff×Q(ta)), for example. The sweat intensity S may be represented by an expression different from the expression described above. The sweat intensity estimation section 23 sets the derived sweat intensity S as the sweat amount data 20A (to be included in the sweat amount data 20A).

The sweat intensity estimation section 23 outputs the sweat amount data 20A generated as described above to the data output unit 40. The data output unit 40 outputs the sweat amount data 20A acquired from the sweat intensity estimation section 23 to the outside. For example, every time a time region with high reliability is detected by the reliability determination section 22 in the internal sweat amount data Q(t) and the external sweat amount data C(t), the sweat intensity estimation section 23 derives the sweat intensity S (step S204; N). For example, when a predetermined condition is satisfied, the sweat intensity estimation section 23 finishes the derivation of the sweat intensity S (Step S204; Y)

### [Effects]

Next description is given of effects of the biological information processor 1.

In the biological information processor 1, the sweat amount data 20A to be outputted to the outside is generated on the basis of the external sweat amount data C(t) and the internal sweat amount data Q(t) generated on the basis of the outputs of the first sensor section 11 and the second sensor section 12. This makes it possible, for example, to use the internal sweat amount data Q(t) as reference data of the external sweat amount data C(t) and to use the external sweat amount data C(t) as reference data of the internal sweat amount data Q(t). As a result, it is possible to detect and reduce a body motion noise from the external sweat amount data C(t) or the internal sweat amount data Q(t) without using an output of the pressure sensor as the reference data. Thus, it is possible to reduce the body motion noise more effectively.

In the biological information processor 1, the reliability of the external sweat amount data C(t) is evaluated on the basis of the second difference data ΔQ(t), which is a difference between two pieces of data having detection times different from each other, among the internal sweat amount data Q(t). Then, on the basis of a result of the evaluation of the reliability of the external sweat amount data C(t), processing is performed on the external sweat amount data C(t), and the resulting data serves as the sweat amount data 20A. In this manner, it is possible to detect and reduce the body motion noise from the external sweat amount data C(t) without using the output of the pressure sensor as the reference data. Thus, it is possible to reduce the body motion noise more effectively.

In the biological information processor 1, the reliability of the internal sweat amount data Q(t) is evaluated on the basis of the first difference data ΔC(t), which is a difference between two pieces of data having detection times different from each other, among the external sweat amount data C(t). Then, on the basis of a result of the evaluation of the reliability of the internal sweat amount data Q(t), processing is performed on the internal sweat amount data Q(t), and the resulting data serves as the sweat amount data 20A. In this manner, it is possible to detect and reduce the body motion noise from the internal sweat amount data Q(t) without using the output of the pressure sensor as the reference data. Thus, it is possible to reduce the body motion noise more effectively.

In the biological information processor 1, in a case where the second difference data ΔQ(t) and the first difference data ΔC(t) include a correlation of a reverse phase with respect to each other, each reliability of portions in the correlation of the reverse phase with respect to each other, among the external sweat amount data C(t) and the internal sweat amount data Q(t), is given a low evaluation. Further, in a case where the second difference data ΔQ(t) and the first difference data ΔC(t) include a correlation of an in phase with respect to each other, each reliability of portions in the correlation of the in phase with respect to each other, among the external sweat amount data C(t) and the internal sweat amount data Q(t), is given a high evaluation. Then, the data of the portions evaluated to have high reliability, among the external sweat amount data C(t) and the internal sweat amount data Q(t), serves as the sweat amount data 20A, and the data of the portions evaluated to have low reliability, among the external sweat amount data C(t) and the internal sweat amount data Q(t), is excluded from the sweat amount data 20A. In this manner, only the data evaluated to have high reliability remains as the sweat amount data 20A to be outputted to the outside. Thus, it is possible to provide the sweat amount data 20A in which the body motion noise is reduced more effectively.

In the biological information processor 1, the reliability of the external sweat amount data C(t) is evaluated on the basis of the second difference data ΔQ(t), which is a difference between two pieces of data having detection times different from each other, among the internal sweat amount data Q(t). Then, on the basis of a result of the evaluation of the reliability of the external sweat amount data C(t), reliability evaluation data Ex(t) on the external sweat amount data C(t) is generated. This makes it possible to extract data with high reliability from among data included in the sweat amount data 20A by referring to the reliability evaluation data Ex(t), without excluding data evaluated to have low reliability from the sweat amount data 20A to be outputted to the outside. Thus, outputting the reliability evaluation data Ex(t) together with the external sweat amount data C(t) to the outside makes it possible to provide the sweat amount data 20A in which the body motion noise is reduced more effectively.

In the biological information processor 1, the reliability of the internal sweat amount data Q(t) is evaluated on the basis of the first difference data ΔQ(t), which is a difference between two pieces of data having detection times different from each other, among the external sweat amount data C(t). Then, on the basis of a result of the evaluation of the reliability of the internal sweat amount data Q(t), the reliability evaluation data Eq(t) on the internal sweat amount data Q(t) is generated. This makes it possible to extract data with high reliability from among data included in the sweat amount data 20A by referring to the reliability evaluation data Eq(t), without excluding data evaluated to have low reliability from the sweat amount data 20A to be outputted to the outside. Thus, outputting the reliability evaluation data Eq(t) together with the internal sweat amount data Q(t) to the outside makes it possible to provide the sweat amount data 20A in which the body motion noise is reduced more effectively.

### 2. Modification Example

In the foregoing embodiment, for example, as illustrated in FIG. 12, a processing program 31 may be stored in the storage unit 30. At this time, the processing program 31 may be loaded into the signal processing unit 20 to thereby cause the signal processing unit 20 to execute the various functions described above. Also in such a case, it is possible to obtain effects similar to those of the foregoing embodiment.

### 3. Application Example

Hereinafter, description is given of an example in which the biological information processor 1 according to the foregoing embodiment and modification example thereof is applied to a wearable apparatus 200.

FIG. 13 illustrates a state in which the biological information processor 1 according to the foregoing embodiment and modification example thereof is built in the wearable apparatus 200. FIG. 13 illustrates a state in which the biological information processor 1 is built in the wearable apparatus 200 of a wristband type or a wristwatch type. FIG. 14 illustrates an example of a side configuration of the wearable apparatus 200 in FIG. 13. The wearable apparatus 200 includes, for example, a housing 210 that accommodates various devices, and a belt 220 to fix a wrist. The housing 210 accommodates, for example, the signal processing unit 20, the storage unit 30, and the data output unit 40. For example, the sensor unit 10 is provided on an inner surface of the belt 220. For example, the detection electrode 11A and the light source 12A are provided at a location, of the sensor unit 10, in contact with a wrist of the biological body 100.

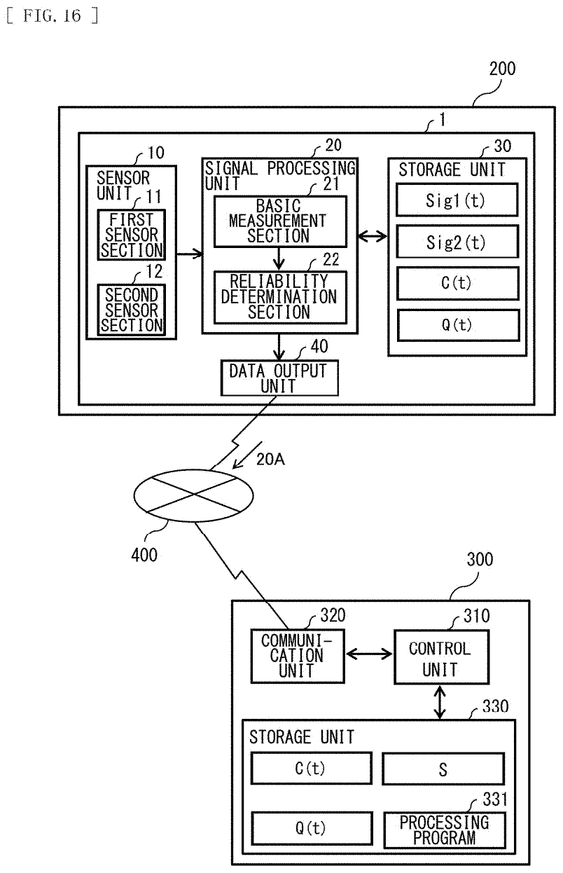

The biological information processor 1 built in the wearable apparatus 200 is coupled to a terminal apparatus 300 via a network 400, for example, as illustrated in FIG. 15. At this time, in the biological information processor 1 built in the wearable apparatus 200, the data output unit 40 is coupled to the terminal apparatus 300 via the network 400, for example, as illustrated in FIG. 16. In the biological information processor 1 built in the wearable apparatus 200, the signal processing unit 20 includes the basic measurement section 21 and the reliability determination section 22. The sweat intensity estimation section 23 is omitted from the signal processing unit 20, and a processing program 331 that implements the functions of the sweat intensity estimation section 23 is stored in a storage unit 330 of the terminal apparatus 300. That is, the functions of the sweat intensity estimation section 23 are transferred from the biological information processor 1 to the terminal apparatus 300. In the biological information processor 1 built in the wearable apparatus 200, the storage unit 30 stores the sensor data (first sensor data Sig1(t) and second sensor data Sig2(t)) obtained from the sensor unit 10 and the data (external sweat amount data C(t) and internal sweat amount data Q(t)) generated by the signal processing unit 20.

The terminal apparatus 300 includes, for example, a control unit 310, a communication unit 320, and the storage unit 330. Loading the processing program 331 into the control unit 310 causes the control unit 310 to execute the functions of the sweat intensity estimation section 23. The storage unit 330 is, for example, a volatile memory such as a DRAM, or a non-volatile memory such as an EEPROM or a flash memory. The storage unit 330 stores data (external sweat amount data C(t) and internal sweat amount data Q(t)) transmitted from the biological information processor 1 built in the wearable apparatus 200, and data (sweat intensity S) obtained by execution of the processing program 331 in the control unit 310.

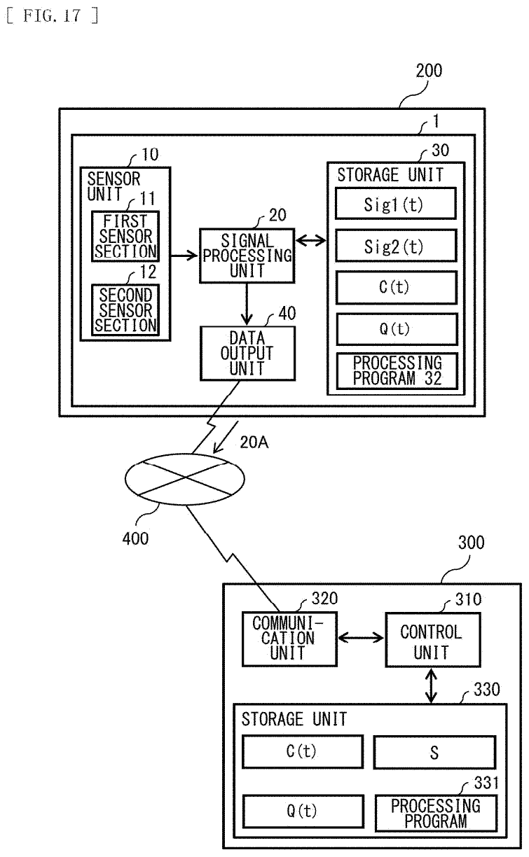

It is to be noted that, for example, as illustrated in FIG. 17, a processing program 32 may be stored in the storage unit 30. At this time, the processing program 32 may be loaded into the signal processing unit 20 to thereby cause the signal processing unit 20 to execute the functions of the basic measurement section 21 and the reliability determination section 22. Also in such a case, it is possible to obtain effects similar to those of the foregoing embodiment.

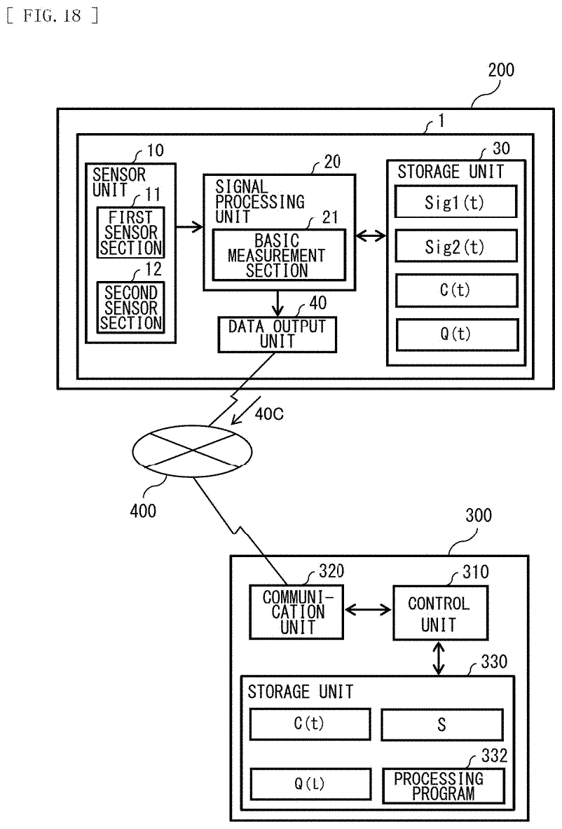

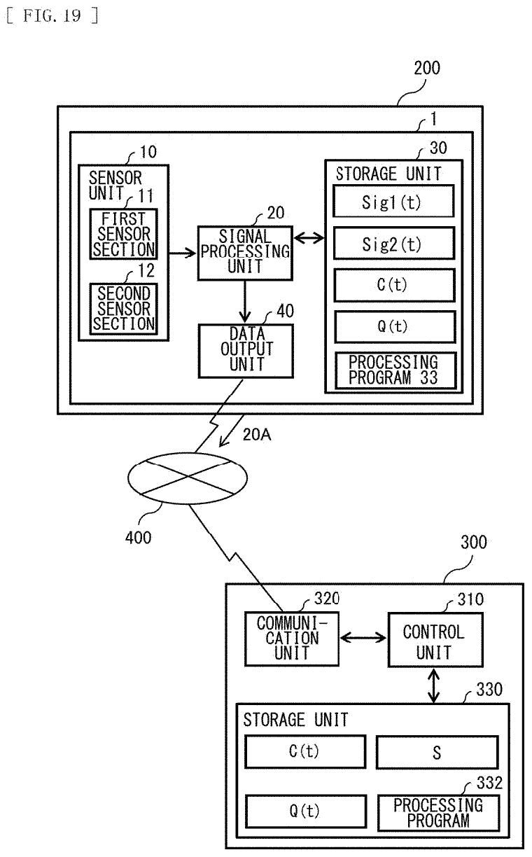

In addition, in the biological information processor 1 built in the wearable apparatus 200, the signal processing unit 20 may include, for example, the basic measurement section 21 as illustrated in FIG. 18. At this time, the reliability determination section 22 and the sweat intensity estimation section 23 are omitted from the signal processing unit 20, and a processing program 332 that implements the functions of the reliability determination section 22 and the sweat intensity estimation section 23 is stored in the storage unit 330 of the terminal apparatus 300. That is, the functions of the reliability determination section 22 and the sweat intensity estimation section 23 are transferred from the biological information processor 1 to the terminal apparatus 300. In the biological information processor 1 built in the wearable apparatus 200, the storage unit 30 stores the sensor data (first sensor data Sig1(t) and second sensor data Sig2(t)) obtained from the sensor unit 10 and the data (external sweat amount data C(t) and internal sweat amount data Q(t)) generated by the signal processing unit 20.

The terminal apparatus 300 includes, for example, the control unit 310, the communication unit 320, and the storage unit 330. Loading the processing program 332 into the control unit 310 causes the control unit 310 to execute the functions of the reliability determination section 22 and the sweat intensity estimation section 23. The storage unit 330 stores the data (external sweat amount data C(t) and internal sweat amount data Q(t)) transmitted from the biological information processor 1 built in the wearable apparatus 200, and the data (sweat intensity S) obtained by execution of the processing program 332 in the control unit 310.

It is to be noted that, for example, as illustrated in FIG. 19, a processing program 33 may be stored in the storage unit 30. At this time, the processing program 33 may be loaded into the signal processing unit 20 to thereby cause the signal processing unit 20 to execute the functions of the basic measurement section 21. Also in such a case, it is possible to obtain effects similar to those of the foregoing embodiment.

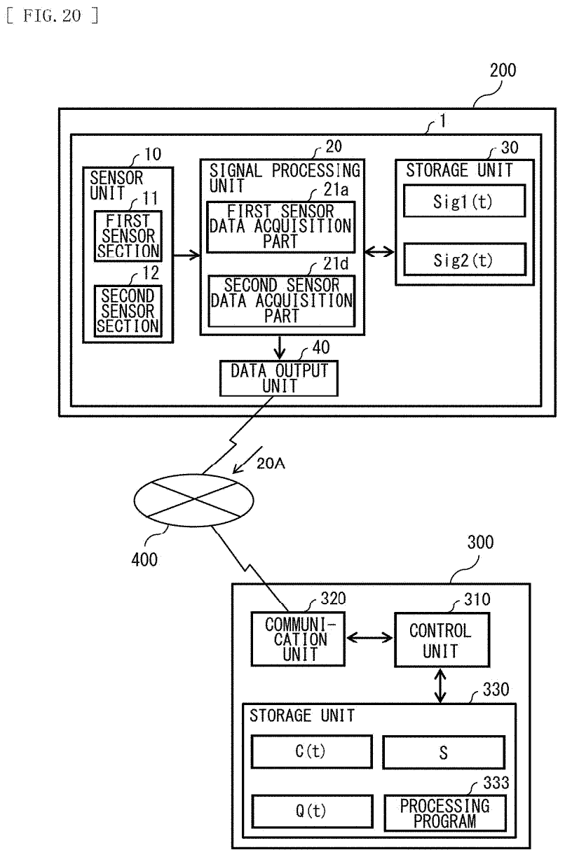

In addition, in the biological information processor 1 built in the wearable apparatus 200, the signal processing unit 20 may include, for example, the first sensor data acquisition part 21a and the second sensor data acquisition part 21d as illustrated in FIG. 20. At this time, the external sweat amount calculation part 21b, the variation calculation part 21c, the internal sweat amount calculation part 21e, the variation calculation part 21f, the reliability determination section 22, and the sweat intensity estimation section 23 are omitted from the signal processing unit 20, and a processing program 333 that implements the functions of the external sweat amount calculation part 21b, the variation calculation part 21c, the internal sweat amount calculation part 21e, the variation calculation part 21f, the reliability determination section 22, and the sweat intensity estimation section 23 is stored in the storage unit 330 of the terminal apparatus 300. That is, the functions of the external sweat amount calculation part 21b, the variation calculation part 21c, the internal sweat amount calculation part 21e, the variation calculation part 21f, the reliability determination section 22, and the sweat intensity estimation section 23 are transferred from the biological information processor 1 to the terminal apparatus 300. In the biological information processor 1 built in the wearable apparatus 200, the storage unit 30 stores the sensor data (first sensor data Sig1(t) and second sensor data Sig2(t)) obtained from the sensor unit 10.

The terminal apparatus 300 includes, for example, the control unit 310, the communication unit 320, and the storage unit 330. Loading the processing program 333 into the control unit 310 causes the control unit 310 to execute the functions of the external sweat amount calculation part 21b, the variation calculation part 21c, the internal sweat amount calculation part 21e, the variation calculation part 21f, the reliability determination section 22, and the sweat intensity estimation section 23. The storage unit 330 stores the data (external sweat amount data C(t) and internal sweat amount data Q(t)) transmitted from the biological information processor 1 built in the wearable apparatus 200, and the data (sweat intensity S) obtained by execution of the processing program 333 in the control unit 310.

It is to be noted that the effects described herein are merely illustrative. The effects of the present disclosure are not limited to those described herein. The present disclosure may also have effects other than those described herein.

For example, the above-described series of processing may be executed by software or may be executed by hardware.

In addition, the foregoing plurality of embodiments and modification examples thereof are applicable to an application that requires objective cognitive load tolerance, for example, in games, healthcare, learning, training for sports games, training for human resource development, driving of a mobile body such as an automobile, and the like.

In addition, for example, the present disclosure may have the following configurations.

(1)

An information processor including:
an acquisition section that acquires external sweat amount data on an amount of sweat oozing out of an epidermis and internal sweat amount data on an amount of sweat inside the epidermis and a dermis, the external sweat amount data being generated on a basis of an output of a first sensor section, the internal sweat amount data being generated on a basis of an output of a second sensor section; and
a generation section that generates sweat amount data on a basis of the external sweat amount data and the internal sweat amount data.
   (2)

The information processor according to (1), further including an evaluation section that evaluates reliability of the external sweat amount data on a basis of internal sweat amount difference data which is a difference between two pieces of data having detection times different from each other, among the internal sweat amount data, in which
the generation section performs processing on the external sweat amount data on a basis of a result of the evaluation of the reliability of the external sweat amount data by the evaluation section, and sets resulting data as the sweat amount data.
   (3)

The information processor according to (2), in which
the evaluation section evaluates reliability of the internal sweat amount data on a basis of external sweat amount difference data which is a difference between two pieces of data having detection times different from each other, among the external sweat amount data, and
the generation section performs processing on the internal sweat amount data on a basis of a result of the evaluation of the reliability of the internal sweat amount data by the evaluation section, and sets resulting data as the sweat amount data.
   (4)

The information processor according to (3), in which
in a case where the internal sweat amount difference data and the external sweat amount difference data include a correlation of a reverse phase with respect to each other, the evaluation section gives a low evaluation to each reliability of portions in the correlation of the reverse phase with respect to each other, among the external sweat amount data and the internal sweat amount data, and, in a case where the internal sweat amount difference data and the external sweat amount difference data include a correlation of an in phase with respect to each other, the evaluation section gives a high evaluation to each reliability of portions in the correlation of the in phase with respect to each other, among the external sweat amount data and the internal sweat amount data, and
the generation section sets data of the portions evaluated to have high reliability by the evaluation section, among the external sweat amount data and the internal sweat amount data, as the sweat amount data, and excludes data of the portions evaluated to have low reliability by the evaluation section, among the external sweat amount data and the internal sweat amount data, from the sweat amount data.
   (5)

The information processor according to (4), further including an output section that outputs, to an outside, the sweat amount data in which the evaluation by the evaluation section is reflected.

(6)

The information processor according to (1), further including an evaluation section that evaluates reliability of the external sweat amount data on a basis of internal sweat amount difference data which is a difference between two pieces of data having detection times different from each other, among the internal sweat amount data, in which
the generation section generates first reliability evaluation data on the external sweat amount data on a basis of a result of the evaluation of the reliability of the external sweat amount data by the evaluation section.
   (7)

The information processor according to (6), further including an output section that outputs, as the sweat amount data, the external sweat amount data and the first reliability evaluation data to an outside.

(8)

The information processor according to (6), in which
the evaluation section evaluates reliability of the internal sweat amount data on a basis of external sweat amount difference data which is a difference between two pieces of data having detection times different from each other, among the external sweat amount data, and
the generation section generates second reliability evaluation data on the internal sweat amount data on a basis of a result of the evaluation of the reliability of the internal sweat amount data by the evaluation section.
   (9)

The information processor according to (8), further including an output section that outputs, as the sweat amount data, the external sweat amount data, the internal sweat amount data, the first reliability evaluation data, and the second reliability evaluation data to an outside.

(10)

The information processor according to any one of (1) to (9), further including:
the first sensor section that detects an external sweat amount, and outputs data thus obtained; and
the second sensor section that detects an internal sweat amount, and outputs data thus obtained.
   (11)

The information processor according to (10), in which
the first sensor section electrically detects the external sweat amount, and
the second sensor section optically detects the internal sweat amount.
   (12)

An information processing program that causes a computer to:
acquire external sweat amount data generated on a basis of an output of a first sensor section and internal sweat amount data generated on a basis of an output of a second sensor; and
generate sweat amount data on a basis of the external sweat amount data and the internal sweat amount data.

According to the information processor of a first aspect of the present disclosure, sweat amount data is generated on the basis of external sweat amount data and internal sweat amount data generated on the basis of outputs of the first sensor section and the second sensor section, thus making it possible, for example, to use the internal sweat amount data as reference data of the external sweat amount data and to use the external sweat amount data as reference data of the internal sweat amount data. As a result, it is possible to reduce a body motion noise from the external sweat amount data or the internal sweat amount data without using the output of the pressure sensor as the reference data. Thus, it is possible to reduce the body motion noise effectively.

According to the information processing program of a second aspect of the present disclosure, the sweat amount data is generated on the basis of the external sweat amount data and the internal sweat amount data generated on the basis of outputs of the first sensor section and the second sensor section, thus making it possible, for example, to use the internal sweat amount data as reference data of the external sweat amount data and to use the external sweat amount data as reference data of the internal sweat amount data. As a result, it is possible to reduce a body motion noise from the external sweat amount data or the internal sweat amount data without using the output of the pressure sensor as the reference data. Thus, it is possible to reduce the body motion noise effectively.

In addition, for example, the present disclosure may have the following configurations.

(1)

An information processor including:
an acquisition section that acquires first sweat amount data generated on a basis of an output of an electric sensor section and second sweat amount data generated on a basis of an output of an optical sensor section; and
a generation section that generates sweat amount data on a basis of the first sweat amount data and the second sweat amount data.
   (2)

The information processor according to (1), further including an evaluation section that evaluates reliability of the first sweat amount data on a basis of second sweat amount difference data which is a difference between two pieces of data having detection times different from each other, among the second sweat amount data, in which
the generation section performs processing on the first sweat amount data on a basis of a result of the evaluation of the reliability of the first sweat amount data by the evaluation section, and sets resulting data as the sweat amount data.
   (3)

The information processor according to (2), in which
the evaluation section evaluates reliability of the second sweat amount data on a basis of first sweat amount difference data which is a difference between two pieces of data having detection times different from each other, among the first sweat amount data, and
the generation section performs processing on the second sweat amount data on a basis of a result of the evaluation of the reliability of the second sweat amount data by the evaluation section, and sets resulting data as the sweat amount data.
   (4)

The information processor according to (3), in which
in a case where the second sweat amount difference data and the first sweat amount difference data include a correlation of a reverse phase with respect to each other, the evaluation section gives a low evaluation to each reliability of portions in the correlation of the reverse phase with respect to each other, among the first sweat amount data and the second sweat amount data, and, in a case where the second sweat amount difference data and the first sweat amount difference data include a correlation of an in phase with respect to each other, the evaluation section gives a high evaluation to each reliability of portions in the correlation of the in phase with respect to each other, among the first sweat amount data and the second sweat amount data, and
the generation section sets data of the portions evaluated to have high reliability by the evaluation section, among the first sweat amount data and the second sweat amount data, as the sweat amount data, and excludes data of the portions evaluated to have low reliability by the evaluation section, among the first sweat amount data and the second sweat amount data, from the sweat amount data.
   (5)

The information processor according to (4), further including an output section that outputs, to an outside, the sweat amount data in which the evaluation by the evaluation section is reflected.

(6)

The information processor according to (1), further including an evaluation section that evaluates reliability of the first sweat amount data on a basis of second sweat amount difference data which is a difference between two pieces of data having detection times different from each other, among the second sweat amount data, in which
the generation section generates first reliability evaluation data on the first sweat amount data on a basis of a result of the evaluation of the reliability of the first sweat amount data by the evaluation section.
   (7)

The information processor according to (6), further including an output section that outputs, as the sweat amount data, the first sweat amount data and the first reliability evaluation data to an outside.

(8)

The information processor according to (6), in which
the evaluation section evaluates reliability of the second sweat amount data on a basis of first sweat amount difference data which is a difference between two pieces of data having detection times different from each other, among the first sweat amount data, and
the generation section generates second reliability evaluation data on the second sweat amount data on a basis of a result of the evaluation of the reliability of the second sweat amount data by the evaluation section.
   (9)

The information processor according to (8), further including an output section that outputs, as the sweat amount data, the first sweat amount data, the second sweat amount data, the first reliability evaluation data, and the second reliability evaluation data to an outside.

(10)

The information processor according to any one of (1) to (9), further comprising:
the electric sensor section that electrically detects an amount of sweat oozing out of an epidermis; and
the optical sensor section that optically detects an amount of sweat inside the epidermis and a dermis.
   (11)

An information processing program that causes a computer to:
acquire first sweat amount data generated on a basis of an output of an electric sensor section and second sweat amount data generated on a basis of an output of an optical sensor; and
generate sweat amount data on a basis of the first sweat amount data and the second sweat amount data.

According to the information processor of a first aspect of the present disclosure, sweat amount data is generated on the basis of first sweat amount data and second sweat amount data generated on the basis of outputs of the electric sensor section and the optical sensor section, thus making it possible, for example, to use the second sweat amount data as reference data of the first sweat amount data and to use the first sweat amount data as reference data of the second sweat amount data. As a result, it is possible to detect and reduce a body motion noise from the first sweat amount data or the second sweat amount data without using the output of the pressure sensor as the reference data. Thus, it is possible to reduce the body motion noise more effectively.

According to the information processing program of a second aspect of the present disclosure, the sweat amount data is generated on the basis of the first sweat amount data and the second sweat amount data generated on the basis of outputs of the electric sensor section and the optical sensor section, thus making it possible, for example, to use the second sweat amount data as reference data of the first sweat amount data and to use the first sweat amount data as reference data of the second sweat amount data. As a result, it is possible to detect and reduce a body motion noise from the first sweat amount data or the second sweat amount data without using the output of the pressure sensor as the reference data. Thus, it is possible to reduce the body motion noise more effectively.

This application claims the benefits of Japanese Priority Patent Application JP2020-072405 filed with the Japan Patent Office on Apr. 14, 2020, the entire contents of which are incorporated herein by reference.

It should be understood by those skilled in the art that various modifications, combinations, sub-combinations, and alterations may occur depending on design requirements and other factors insofar as they are within the scope of the appended claims or the equivalents thereof.

1. An information processor comprising:
an acquisition section that acquires external sweat amount data on an amount of sweat oozing out of an epidermis and internal sweat amount data on an amount of sweat inside the epidermis and a dermis, the external sweat amount data being generated on a basis of an output of a first sensor section, the internal sweat amount data being generated on a basis of an output of a second sensor section; and
a generation section that generates sweat amount data on a basis of the external sweat amount data and the internal sweat amount data.

2. The information processor according to claim 1, further comprising an evaluation section that evaluates reliability of the external sweat amount data on a basis of internal sweat amount difference data which is a difference between two pieces of data having detection times different from each other, among the internal sweat amount data, wherein
the generation section performs processing on the external sweat amount data on a basis of a result of the evaluation of the reliability of the external sweat amount data by the evaluation section, and sets resulting data as the sweat amount data.

3. The information processor according to claim 2, wherein
the evaluation section evaluates reliability of the internal sweat amount data on a basis of external sweat amount difference data which is a difference between two pieces of data having detection times different from each other, among the external sweat amount data, and
the generation section performs processing on the internal sweat amount data on a basis of a result of the evaluation of the reliability of the internal sweat amount data by the evaluation section, and sets resulting data as the sweat amount data.

4. The information processor according to claim 3, wherein
in a case where the internal sweat amount difference data and the external sweat amount difference data include a correlation of a reverse phase with respect to each other, the evaluation section gives a low evaluation to each reliability of portions in the correlation of the reverse phase with respect to each other, among the external sweat amount data and the internal sweat amount data, and, in a case where the internal sweat amount difference data and the external sweat amount difference data include a correlation of an in phase with respect to each other, the evaluation section gives a high evaluation to each reliability of portions in the correlation of the in phase with respect to each other, among the external sweat amount data and the internal sweat amount data, and
the generation section sets data of the portions evaluated to have high reliability by the evaluation section, among the external sweat amount data and the internal sweat amount data, as the sweat amount data, and excludes data of the portions evaluated to have low reliability by the evaluation section, among the external sweat amount data and the internal sweat amount data, from the sweat amount data.

5. The information processor according to claim 4, further comprising an output section that outputs, to an outside, the sweat amount data in which the evaluation by the evaluation section is reflected.

6. The information processor according to claim 1, further comprising an evaluation section that evaluates reliability of the external sweat amount data on a basis of internal sweat amount difference data which is a difference between two pieces of data having detection times different from each other, among the internal sweat amount data, wherein
the generation section generates first reliability evaluation data on the external sweat amount data on a basis of a result of the evaluation of the reliability of the external sweat amount data by the evaluation section.

7. The information processor according to claim 6, further comprising an output section that outputs, as the sweat amount data, the external sweat amount data and the first reliability evaluation data to an outside.

8. The information processor according to claim 6, wherein
the evaluation section evaluates reliability of the internal sweat amount data on a basis of external sweat amount difference data which is a difference between two pieces of data having detection times different from each other, among the external sweat amount data, and
the generation section generates second reliability evaluation data on the internal sweat amount data on a basis of a result of the evaluation of the reliability of the internal sweat amount data by the evaluation section.

9. The information processor according to claim 8, further comprising an output section that outputs, as the sweat amount data, the external sweat amount data, the internal sweat amount data, the first reliability evaluation data, and the second reliability evaluation data to an outside.

10. The information processor according to claim 1 further comprising:
the first sensor section that detects an external sweat amount, and outputs data thus obtained; and
the second sensor section that detects an internal sweat amount, and outputs data thus obtained.

11. The information processor according to claim 10, wherein
the first sensor section electrically detects the external sweat amount, and
the second sensor section optically detects the internal sweat amount.

12. An information processing program that causes a computer to:
acquire external sweat amount data on an amount of sweat oozing out of an epidermis and internal sweat amount data on an amount of sweat inside the epidermis and a dermis, the external sweat amount data being generated on a basis of an output of a first sensor section, the internal sweat amount data being generated on a basis of an output of a second sensor; and
generate sweat amount data on a basis of the external sweat amount data and the internal sweat amount data.
